(19) United States
(12) Patent Application Publication
(10) Pub. No.: US 2025/0318773 A1
(43) Pub. Date: Oct. 16, 2025

(54) SYSTEM AND METHOD FOR EARLY DETECTION OF COGNITIVE IMPAIRMENT USING COGNITIVE TEST RESULTS WITH ITS BEHAVIORAL METADATA

(71) Applicant: OHIO STATE INNOVATION FOUNDATION, Columbus, OH (US)

(72) Inventors: Xia NING, Dublin, OH (US); Douglas SCHARRE, Columbus, OH (US); Ryoma KAWAKAMI, MCLean, VA (US)

(21) Appl. No.: 18/866,675

(22) PCT Filed: May 17, 2023
(86) PCT No.: PCT/US2023/022552

§ 371 Date: Nov. 18, 2024

Related U.S. Application Data
(60) Provisional application No. 63/342,936, filed on May 17, 2022.

(51) Int. Cl. A61B 5/00; A61B 5/11; G06T 7/00; G06T 7/12; G06T 7/60; G06T 7/70; G16H 10/60; G16H 50/30

(52) U.S. Cl. CPC A61B 5/4088; A61B 5/1124; G06T 7/0012; G06T 7/12; G06T 7/60; G06T 7/70; G16H 50/30; A61B 2503/20; G06T 2207/30242; G16H 10/60

(57) ABSTRACT

An exemplary system and method are disclosed that is configured to detect cognitive impairment (e.g., early cognitive impairment) or assess cognitive function by analyzing, via machine learning and artificial intelligence analysis, behavioral metadata collected from a smart app during the course when a subject is using a cognitive test instrument for cognitive tests that incorporate motor activity (e.g., drawing or writing). The machine learning and artificial intelligence analysis can execute features associated with the test taker's metadata (e.g., time spent on task or questions, changing answers, referring back to the previous question), drawing qualities (e.g., line straightness, completeness), among others.

### BACKGROUND

Cognitive decline and Alzheimer's Disease (AD) are prevalent among elderly people. Detection of cognitive decline and impairment at an early stage, before the onset of significant symptoms or diagnoses, is important in order to allow for more proactive management and treatment to slow down the progress of cognitive decline or AD.

### SUMMARY

An exemplary system and method are disclosed that is configured to detect cognitive impairment (e.g., early cognitive impairment) or assess cognitive function by analyzing, via machine learning and artificial intelligence analysis, behavioral metadata collected from a smart app during the course when a subject is using a cognitive test instrument for cognitive tests that require motor activity (e.g., drawing or writing). The machine learning and artificial intelligence analysis can execute features associated with the test taker's metadata (e.g., time spent on task or questions, changing answers, referring back to the previous question), drawing qualities (e.g., line straightness, completeness), etc.

The exemplary artificial intelligence (AI) system and methods can be employed to predict cognitive impairment. The system and method employ, cognitive tests scoring and results data and metadata features extracted during the cognitive test (behavior data acquired and/or logged during the test) with or without including electronic medical record data, to accurately estimate or predict the presence or non-presence of cognitive impairment from. As used herein, the term “predict” refers to an estimation or a determination of a likelihood of a condition of interest.

It was observed that the metadata feature can also provide increased accuracy performance to prediction/estimations using cognitive tests results alone.

In an aspect, a method is disclosed to assess cognitive impairment or cognitive function, the method comprising: obtaining, by one or more processors, a first data set comprising a set of question scores for a set of cognitive questions performed by a user; obtaining, by the one or more processors, a second data set comprising at least one of a timing component log, a writing component log, and a drawing component log acquired during completion of the at least one of the set of cognitive questions by the user; determining, by the one or more processors utilizing at least a portion of the first data set and second data set, one or more calculated values for at least one of a timing component feature, a writing component feature, and a drawing component feature for each of the at least one of the set of cognitive questions, wherein the drawing component feature includes at least one of a number of strokes, a total length of strokes, an average length of strokes per stroke, an average speed of strokes per stroke, an average straightness per stroke, a geometric area assessment of the strokes, or a geometric perimeter assessment of the strokes; determining, by the one or more processors, based on the one or more calculated values for the at least one of the timing component feature, the writing component feature, and the drawing component feature, an estimated value for a presence of a cognitive condition or a score for cognitive level function; and outputting, via a report and/or display, (i) the estimated value for the presence of the cognitive condition or an indicator of either or (ii) the score for cognitive level function, wherein the output is made available to a healthcare provider, a test evaluator, or a user to assist in a diagnosis of a cognitive condition or a quantification of cognitive function.

In some embodiments, the estimated value for a presence of a cognitive condition or a score for the cognitive level function is determined using one or more trained ML models.

In some embodiments, the one or more trained ML model includes one or more logistic regression-associated models, one or more support vector machines, one or more neural networks, and/or one or more gradient boost-associated models.

In some embodiments, the estimated value for a presence of a cognitive condition or a score for the cognitive level function is determined using one or more trained AI models.

In some embodiments, the at least one of the timing, writing, and drawing component feature is determined from a time and position log of a user input to a pre-defined writing or drawing area during the completion of the at least one of the set of cognitive questions by the user.

In some embodiments, the drawing component feature is determined by a drawing component analysis module, the drawing component analysis module being configured by computer-readable instructions to: i) identify, for each instance in the time and position log, an entry position and entry time for a given stroke and an exit position and an exit time for the given stroke and ii) determine a measure from the entry position, entry time, exit position and exit time for the given stroke.

In some embodiments, the measure includes at least one of: i) determining the number of strokes; ii) determining the total length of the strokes by (a) determining a length for each of the strokes and (b) summing the determined length; iii) determine the average length of strokes per stroke by (a) determining a length for each stroke and (b) performing an average operation on the determine lengths; iv) determining the average speed of strokes per stroke by (a) determining a velocity for each stroke using length and time measure for a given stroke and (b) performing an average operation on the determine lengths; v) the average straightness per stroke by determining a ratio of a distance between each endpoint of the stroke to a corresponding length of the stroke; and vi) determining a size of a response comprising the strokes.

In some embodiments, the measure of the average straightness per stroke is further determined by segmenting a single stroke of a geometric shape at the corners of the geometric shape to generate individual strokes for each side of the geometric shape.

In some embodiments, the drawing component analysis module is configured to identify a number of extra strokes, wherein the extra strokes are not employed in the measure determination.

In some embodiments, the method further includes: obtaining, by the one or more processors, a third data set comprising electronic health records of the user; and determining, by the one or more processors utilizing a portion of the third data set, one or more calculated second values for a cognitive impairment feature, wherein the one or more calculated second values for the cognitive impairment feature are used with the one or more calculated values for the one or more of the timing, writing, and drawing component feature to determine the estimated value for the presence of a cognitive condition or the score for cognitive level function.

In some embodiments, the first data and the second data are acquired through web services, wherein the estimated value for the presence of the cognitive condition or cognitive level function is outputted through the web services to be displayed at a client device associated with the user.

In some embodiments, the output includes the estimated value for the presence or non-presence of the cognitive or an indicator of either includes a measure for normal cognition, mild cognitive impairment (MCI), or dementia.

In some embodiments, the output includes the estimated value for the presence or non-presence of the cognitive condition or an indicator of either and is used by the healthcare provider to assist in the diagnosis of the early onset of Alzheimer's, dementia, memory loss, or cognitive impairment.

In some embodiments, the output includes the score for cognitive level function and is used by a test evaluator, in part, to evaluate the user in a job interview, a job-related training, or a job-related assessment.

In another aspect, a system (e.g., analysis system) is disclosed comprising a processor; and a memory having instructions stored thereon, wherein the instructions, when executed by a processor, cause the processor to perform any one of the above-discussed methods.

In some embodiments, the system further includes a cognitive test server configured to present and obtain answers for a set of cognitive questions to the user, wherein the cognitive test server is configured to generate a time and position log for the action of the user when answering the set of cognitive questions.

In another aspect, a non-transitory computer-readable medium is disclosed having instructions stored thereon, wherein the instructions, when executed by the processor, cause the processor to perform any one of the above-discussed methods.

### BRIEF DESCRIPTION OF DRAWINGS

FIGS. 1A-IC show example systems for detecting cognitive impairment or assessing cognitive function using artificial intelligence analysis or machine learning analysis in accordance with illustrative embodiments.

FIGS. 2A-2B show example methods to evaluate and/or generate test scores and metadata features from cognitive test questions.

FIG. 3A-3B show example test questions of a cognitive exam.

FIG. 4 provides a list of example questions from the eSAGE cognitive exam.

### DETAILED SPECIFICATION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

There is a benefit to measuring and improving the detection of cognitive impairment. Early detection of cognitive impairment can help individuals receive timely medical and social interventions to slow down the progression of the condition. Cognitive assessment tests are tools used to evaluate an individual's cognitive abilities, which include memory, attention, language, problem-solving, and perceptual skills. These tests are administered to determine the level of cognitive functioning in various domains, and to identify the severity and nature of any impairment. Some commonly used cognitive assessment tests for measuring cognitive impairment include the Mini-Mental State Examination (MMSE), the Montreal Cognitive Assessment (MoCA), the Clock Drawing Test (CDT), Clinical Dementia Rating (CDR), Addenbrooke's Cognitive Examination (ACE), and Electronic Self-Administered Gerocognitive Exam (eSAGE).

While the results of cognitive assessment tests are useful in their own right, the pending disclosure provides enhanced diagnostic determinations by additionally evaluating metadata of the behaviors of test takers (e.g., the time spent on each question, the frequency and/or count of the user changing answers to test questions, and the speed, accuracy, and consistency of drawn responses, etc.). Combining both test scores and evaluations of behavioral metadata has been found to improve identification of mild cognitive impairment and cognitive impairment.

### Example System

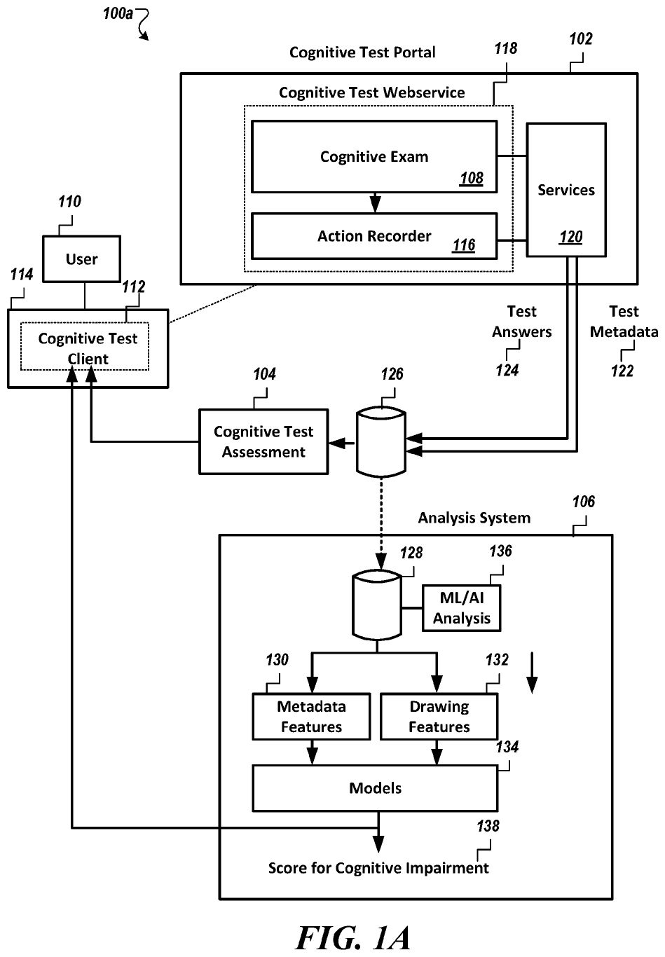

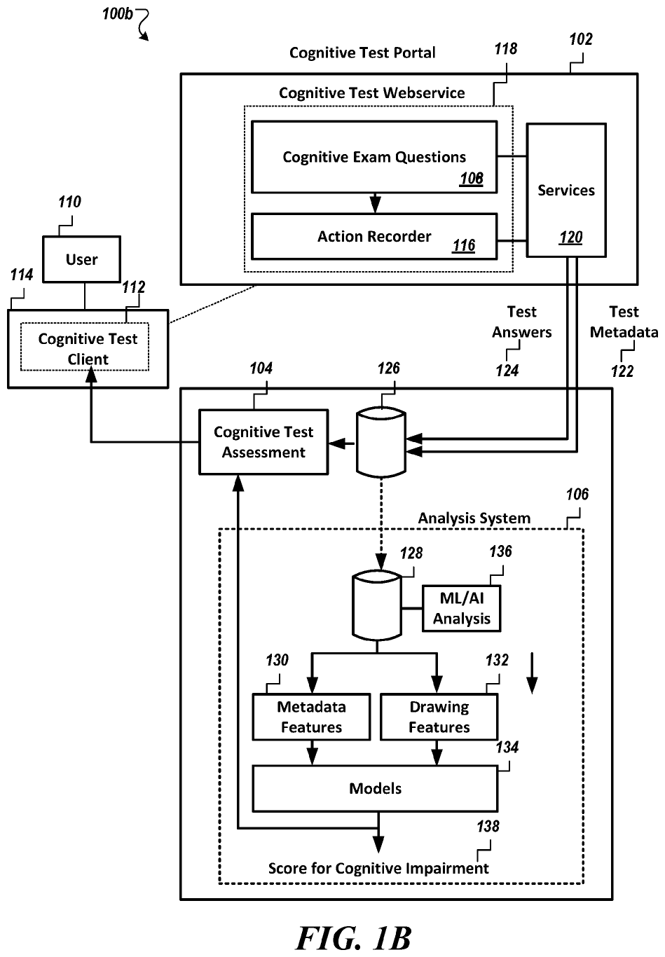

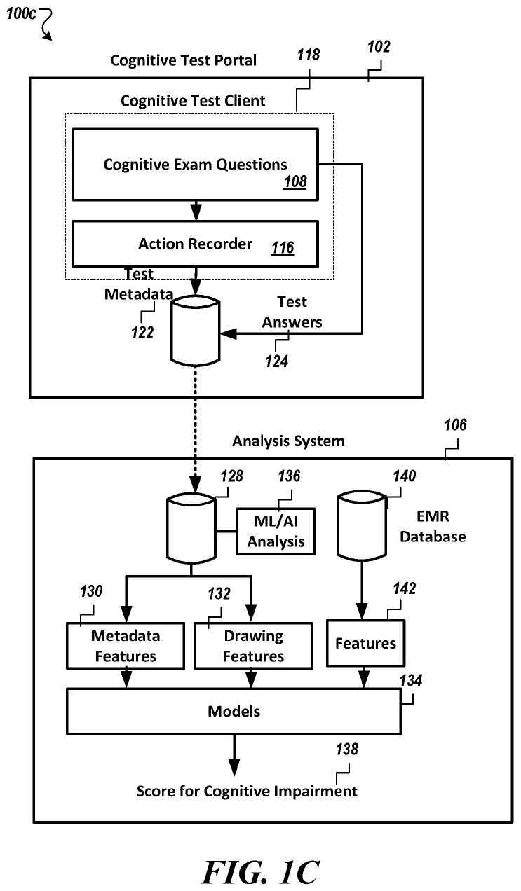

FIG. 1A shows an example system 100a configured to detect cognitive impairment (e.g., early cognitive impairment) or assess cognitive function using artificial intelligence analysis or machine learning analysis in accordance with an illustrative embodiment.

In FIG. 1A, system 100a includes a cognitive test portal 102 (shown as “Cognitive Test Portal”), a test assessment server 104 (shown as “Cognitive Test Assessment” module), and an analysis system 106.

Cognitive Test Portal. The cognitive test portal 102 is configured to administer a cognitive exam 108 with a set of test questions to a user 110 through a client 112 (shown as “Cognitive Test Client”), such as an application executing on a computing device 114 (e.g., a desktop, a laptop, a tablet, a mobile phone, or other personal computing device). While a single user 110 and a single client 112 are shown in the example of FIG. 1A, it is contemplated that the cognitive test portal 102 may simultaneously administer the cognitive exam 108 with a plurality of users on a plurality of computing devices.

An action recorder 116 is configured to record and/or log actions or behaviors performed by the user 110 in answering questions of the cognitive exam 108 during the test. The action recorder 116 stores the recorded information as test metadata 122. In some implementations, one or more (or all) of the test question in the cognitive exam 108 include motor activity (e.g., drawing or writing) for test takers to supply an answer. The action recorder 116 is configured to record aspects of the motor activities performed by the user 110. In various implementations, the action recorder 116 records timestamps with associated actions for evaluating time series behavioral data of the user 110. In various implementations, the action recorder 116 also records locations with associated actions of the user 110. For example, the locations may include a location of the user 110 as well as locations (coordinates) of motor activities performed by the user 110 (e.g., start and stop coordinates of drawn line segments, coordinates of where the user 110 selected within a button relative to the screen or relative to a center or perimeter of a respective button, etc.). The action recorder 116 may also record the selection of buttons or inputs during the test, e.g., changing questions, going to previous questions, etc. The location and time for each of the questions in the cognitive exam 108 and test are stored as the test metadata 122 including one or more behavioral logs or record files of the user 110 during the course of the exam.

In FIG. 1A, a webservice 118 (shown as “Cognitive Test Webservice”) is hosted by the cognitive test portal 102 for supplying the client 112 to the computing device 114. The client 112 may be a standalone application executing on the computing device 114 for administering the cognitive exam 108 and recording behaviors of the user 110 with the action recorder 116. Alternatively or additionally, the client 112 is a thin client for displaying questions of the cognitive exam 108 and/or receiving user input, but the cognitive exam 108 and the action recorder 116 remain at the cognitive test portal 102. Other architectures for how to administer the cognitive exam 108 and receive user input for observation by the action recorder 116 are contemplated by this disclosure.

A services system 120 collects and stores received test metadata 122 and test answers 124 for storage on a cognitive test database 126. The services system 120 interfaces the cognitive test portal 102 to the test assessment server 104, which may be a remote server (e.g., cloud services). The cognitive test portal 102 is configured to transmit (i) the recorded test answers 124 as well as (ii) the recorded test metadata 122 as one or more log files to the test assessment server 104 for analysis.

Cognitive Test Assessment. The test assessment server 104 is configured to receive the test answers 124 from the cognitive test portal 102 and to assess or grade the test answers 124 to a pre-defined test rubric to generate a score for the cognitive exam 108. The test assessment server 104 is configured to assess the answers supplied by the user 110 to questions of the cognitive exam 108, including responses to written or drawn portions of the cognitive exam 108. In some embodiments, the score generated by the test assessment server 104 is employed as a feature for machine learning analysis to evaluate for cognitive impairment (e.g., early cognitive impairment) or assess for cognitive function.

In some implementations, the cognitive test portal 102 and test assessment server 104 may be used to administer and score cognitive performance evaluations or tests relating to a training or education-related activity.

An example of a cognitive test is the eSAGE test [8], [9]. The Self-Administered Gerocognitive Exam (SAGE) is designed to detect early signs of cognitive, memory or thinking impairments. It evaluates your thinking abilities and helps physicians to know how well your brain is working. Other cognitive tests may be similarly administered by the cognitive test portal 102 and scored by the test assessment server 104. For example, the cognitive exam 108 may include any of the Mini-Mental State Examination (MMSE), the Montreal Cognitive Assessment (MoCA), the Clock Drawing Test (CDT), Clinical Dementia Rating (CDR), Addenbrooke's Cognitive Examination (ACE), Electronic Self-Administered Gerocognitive Exam (eSAGE), any combination thereof, or other cognitive examination.

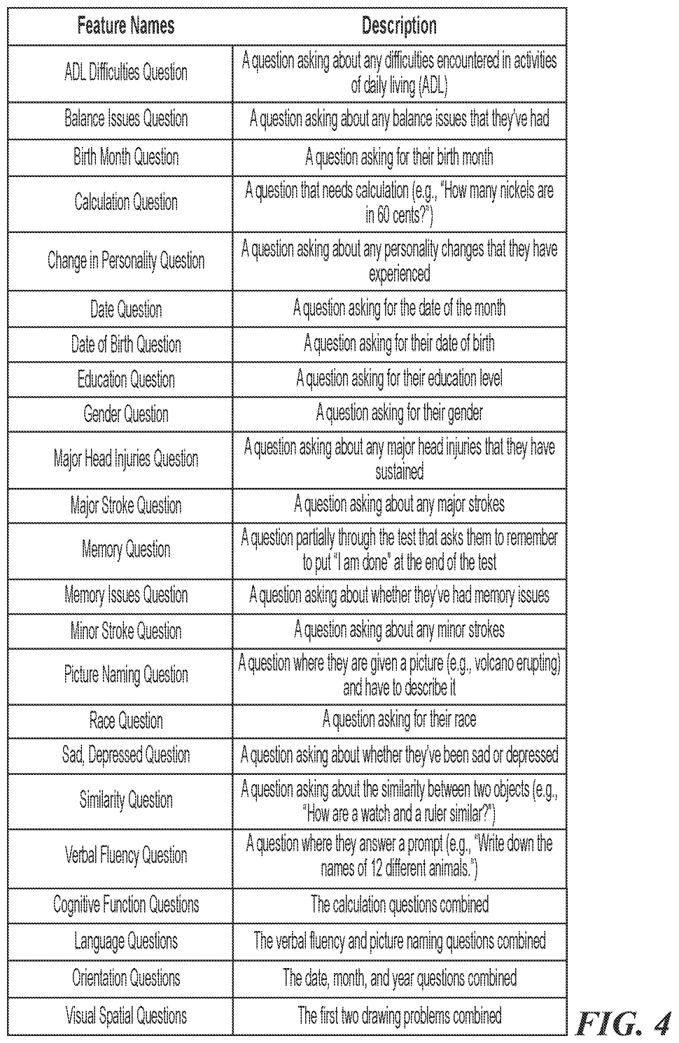

FIG. 4 provides a list of example questions from the eSAGE test. The example questions may include questions relating to i) difficulties in performing daily tasks, ii) balance or dizziness issues, iii) memory questions such as the month of the user's birth, or to recall an action for the end of the test iv) processing questions such a math question, v) personality change questions, vi) past physical injuries to the head, vii) past health issues such as stroke, and the like. The example questions may include handwritten and/or drawing questions that may ask the user 110 to draw and/or write out answers to question.

Analysis System. The analysis system 106 includes artificial intelligence analysis and/or machine learning analysis configured to detect cognitive impairment (e.g., early cognitive impairment) or assess cognitive function.

Metadata Extraction. In FIG. 1A, the analysis system 106 includes a database 128, metadata extraction module 130 (shown as “Metadata Features”), and a drawing component analysis module 132 (shown as a “Drawing Features” module). The metadata extraction module 130 and drawing component analysis module 132 generate behavioral metadata features and drawing features that are input into one or more machine learning models 134 for training the one or more machine learning models 134 and/or determining a cognitive impairment score 138, once trained. In some implementations, the one or more machine learning models 134 also receive the score for the cognitive exam 108 generated by the test assessment server 104 or a score for the cognitive exam 108 generated by the metadata extraction module 130. The cognitive impairment score 138 may be supplied to the client 112 along side or as an alternative to the score supplied by the test assessment server 104.

In various implementations, the metadata extraction module 130 and/or the drawing component analysis module 132 use a behavioral analysis system 136 to extract the metadata features and/or drawing features from the test metadata 122 and/or test answers 124. The behavioral analysis system 136 may be a trained AI or machine learning model for extracting the metadata features and/or drawing features.

The database 128 includes a copy of the records in the cognitive test database 126 or access to records contained within the cognitive test database 126. That is the database 128 stores or has access to the test metadata 122 and the test answers 124.

The metadata extraction module 130 is configured to determine metadata features from behaviors of the user 110 while answering questions of the cognitive exam 108. In some implementations, the metadata extraction module 130 translates raw timestamped button presses or other data of user interface interactions by the user 110 into the metadata features. For example, the metadata extraction module 130 may subtract a difference between time stamps to generate a total time the user 110 spends on a question, such as by taking a difference between timestamps when the user 110 first navigates to a question until they select to navigate to another question. Alternatively or additionally, the metadata extraction module 130 may add together the time the user 110 spends on a question during multiple navigation events to the question based on the user 110 navigating back and forth between questions. In another specific example, the metadata extraction module 130 may determine an amount of time between each button press or between particular sequences of button presses (e.g., time between first button press to input an answer and last button press to complete the answer). More generally, the metadata extraction module 130 may perform any mathematical operation (addition, subtraction, division, multiplication, etc.) or statistical analysis of the test metadata 122 to determine the metadata features.

In some implementations, for each question, the metadata extraction module 130 may determine the score of a given test response, the time spent on each question, as well as frequency and/or count of the user changing answers to the test questions (e.g., timestamps for when the user clicked “next page” or “previous page”). Other metadata features based on user behavior while taking the cognitive exam 108 are contemplated by this disclosure. In some implementations, the metadata extraction module 130 may receive the score of a given test response and/or the score of the cognitive exam 108 from the test assessment server 104. In some implementations, the metadata extraction module 130 may incorporate the functionality of the test assessment server 104 to independently determine the score of a given test response and/or cognitive exam 108.

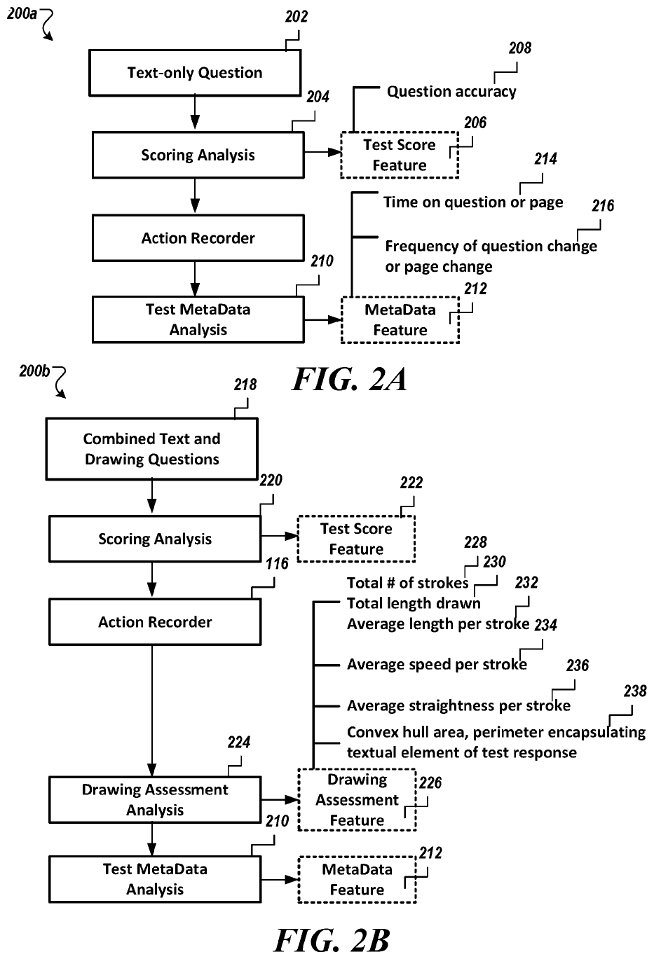

FIG. 2A shows an example method 200a performed by metadata extraction module 130 to evaluate and/or generate test scores and behavioral metadata features from text-only questions.

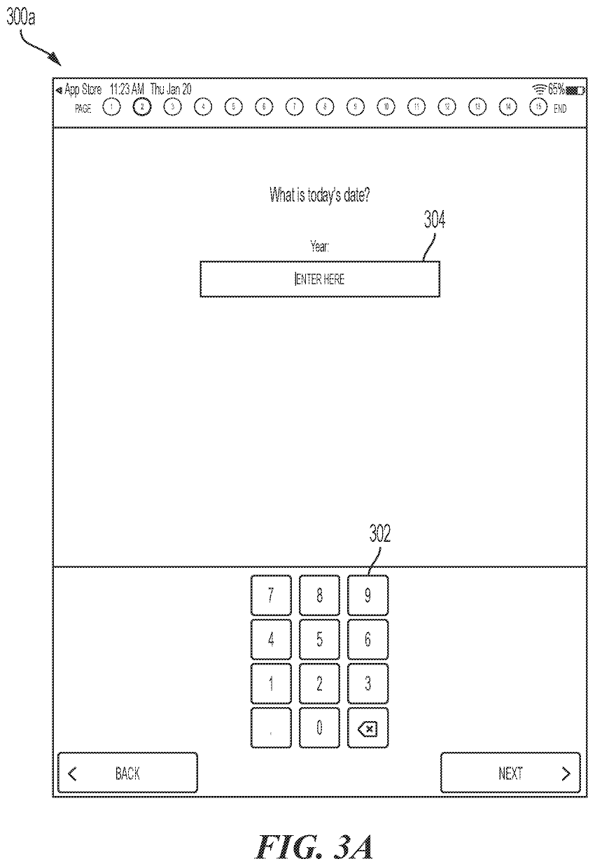

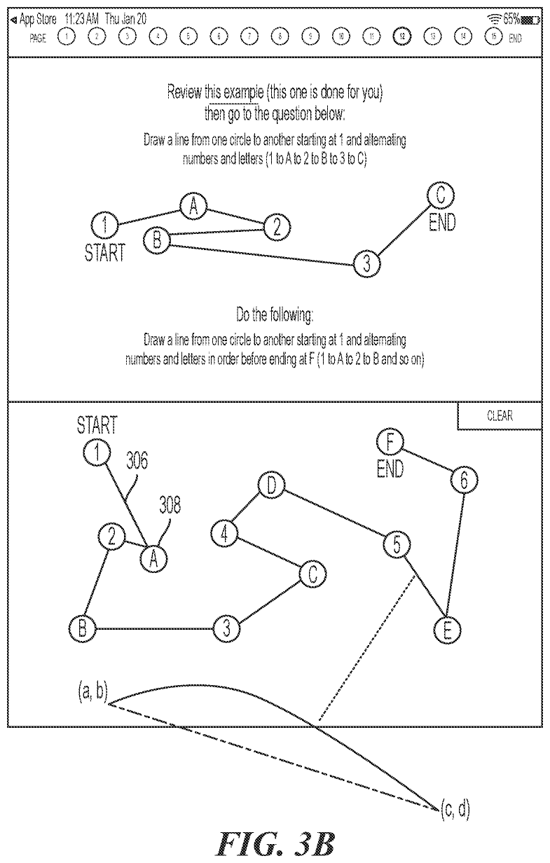

FIG. 3A shows an example of a text-only question 300a. In the example of FIG. 3A, the user 110 is asked to supply the year of today's date. A keypad 302 or other user input device is provided for the user 110 to supply a text answer 304 to the question. While the example shown includes a typed answer, it is contemplated that other text-only questions may include hand written answers. As described above, the action recorder 116 records selections and timing of inputs provided by the user 110 while answering the question 300a. For example, the action recorder 116 may record timestamps associated with each button press and record locations on a touch screen that are selected or simply record which user input (e.g., button) is selected. The action recorder 116 may record all inputs for the duration that the user 110 is working to answer the question, including any selections to delete or change the answer supplied to the question or to navigate to a prior or next question.

Referring back to the method 200a of FIG. 2A, the text-only question 202 is subjected by the metadata extraction module 130 to a scoring or evaluation analysis (shown as “Scoring Analysis 204”) of the provided response to provide a test score 206 for the question 202 (e.g., between 0-2 or between 0-10, etc.) for gauging a question accuracy 208. The question 202 is associated with cognitive impairment or cognitive function. As discussed above, scoring analysis 204 of the metadata extraction module 130 may have functionality similar to the test assessment server 104 for generating the test score 206 or may be in communication with the test assessment server 104 for receiving the test score 206.

In addition, the text-only question 202 is subjected to action recording by the action recorder 116 to provide metadata information to a test metadata analysis 210. The metadata information may additionally include other metadata described above or determined to be useful for gauging cognitive impairment. The test metadata analysis 210 may evaluate the metadata information to generate one or more metadata features 212. In the example shown, the test metadata analysis 210 may generate metadata feature 212 relating to the time that the user spent on the question or page 214 or the frequency that the user changes questions or pages 216. Other metadata features 212, such as those described above, may likewise be generated. In some implementations, the test metadata analysis 210 is performed by or uses the behavioral analysis system 136 to generate the metadata features 212.

Drawing Component Analysis. The analysis system 106 additionally includes a drawing component analysis module 132 (shown as a “Drawing Features” module). The drawing component analysis module 132 is configured to determine metadata features from behaviors of the user 110 from drawn answers to questions of the cognitive exam 108. The drawing component analysis module 132 is configured to identify, for each instance in the time and position log, an entry position and entry time for a given stroke and an exit position and an exit time for the given stroke. The drawing component analysis module 132 may further analyze characteristics of each stroke, the timing and/or sequence of strokes, or other mathematical operation or statistical analysis based on the strokes made by the user 110 in answering questions of the cognitive exam 108.

Though shown in the example of FIG. 1A as separate modules, the metadata extraction module 130 and the drawing component analysis module 132 may be implemented in a single module.

In some implementations, for each question, the drawing component analysis module 132 may determine the score of a given test response, the time spent on each question, as well as frequency and/or count of the user changing answers to the test questions (e.g., timestamps for when the user clicked “next page” or “previous page”). Other metadata features based on user behavior while taking the cognitive exam 108 are contemplated by this disclosure. In some implementations, the drawing component analysis module 132 may receive the score of a given test response and/or the score of the cognitive exam 108 from the test assessment server 104. In some implementations, the drawing component analysis module 132 may incorporate the functionality of the test assessment server 104 to independently determine the score of a given test response and/or cognitive exam 108.

FIG. 2B shows an example method 200b to evaluate and/or generate test scores and metadata features from combined text and drawing questions. The method 200b includes the test metadata analysis 210 and generated metadata features 212 discussed above for text portions of questions.

In FIG. 2B, the combined text and drawing question 218 is subjected by the drawing component analysis module 132 to a scoring analysis 220 to provided responses that provide a test score 222 for the question (e.g., between 0-2 or between 0-10, etc.) that is associated with cognitive impairment or cognitive function. In addition, the combined text and drawing question 218 is additionally subjected to action recording by the action recorder 116 to provide drawing-associated metadata information relating to the speed, accuracy, and consistency of the user's response. As discussed above, the metadata information may include a time and position log of drawn user inputs.

A drawing component analysis module 224 can extract drawing assessment features 226 associated with the speed, accuracy, and consistency of the user's response. The drawing assessment features 226 are calculated from the time and position log of drawn user inputs, including determined entry positions, entry times, exit positions, and exit times for each stroke. The drawing assessment features 226 may be employed as features in a machine learning analysis performed by the one or more machine learning models 134. Example features are provided in Table 1.

TABLE 1

| Type | Desc. |
|---|---|
| Feature 1 | One or more features to determine the number of strokes 228 |
| Feature 2[a . . . n] | One or more features to determine the total length of the strokes 230 |
| | by (a) determining a length for each of the strokes and (b) summing |
| | the determined length. The feature can be determined for each stroke |
| | [a . . . n] in the drawing or handwritten question. |
| Feature 3[a . . . n] | One or more features to determine the average length of strokes per |
| | stroke 232 by (a) determining a length for each stroke and (b) |
| | performing an average operation on the determined lengths. The |
| | feature can be determined for each stroke [a . . . n] in the drawing or |
| | handwritten question. |
| Feature 4[a . . . n] | One or more features to determine the average speed of strokes per |
| | stroke 234 by (a) determining a velocity for each stroke using length |
| | and time measure for a given stroke and (b) performing an average |
| | operation on the determined lengths. The feature can be determined |
| | for each stroke [a . . . n] in the drawing or handwritten question. |
| Feature 5[a . . . n] | One or more features to determine the average straightness per stroke |
| | 236 by determining a ratio of a distance between each endpoint of the |
| | stroke to a corresponding length of the stroke. The feature can be |
| | determined for each stroke [a . . . n] in the drawing or handwritten |
| | question. |
| Feature 6[a . . . n] | One or more features to determine the area and perimeter of a convex |
| | hull around the response for drawing problem 238. |

In some embodiments, the drawing component analysis module 224 is configured to preprocess the time and position log to segment the time and position information into a set of strokes.

FIG. 3B shows an example of a combined text and drawing question excerpted from eSAGE test [8], [9]. The first half of FIG. 3B is an example of a drawing question from eSAGE. In the example shown, the user 110 is instructed to draw various line segments 306 in a particular order between anchor points 308. The second half demonstrates how line straightness is calculated by the drawing component analysis module 224 using data from the time and position log. In the example shown, a measured length of a drawn line is compared to a distance between entry position and exit position for each stroke. Other measures of straightness are contemplated by this disclosure. Other cognitive tests can be similarly employed. FIG. 4 provides a list of example questions from the eSAGE test.

In some embodiments, the analysis system 106 and/or the drawing component analysis module 224 is configured to preprocess the time and position log records or data to identify and filter stray marks and extra strokes. In some implementations, the size, number, or other measure of the stray marks and/or extra strokes may be quantified as features. In some implementations, the stray marks and/or extra strokes may be excluded from the analysis of other features.

In some embodiments, the analysis system 106 and/or the drawing component analysis module 224 is configured to preprocess the time and position log to identify duplicate strokes or repeated strokes.

The analysis system 106 and/or the drawing component analysis module 224 may extract features from the handwriting analysis to be used in the evaluation of cognitive impairment, cognitive conditions or indicators of either.

The analysis system 106 and/or the drawing component analysis module 224, in some embodiments, is configured to generate the one or more machine learning models 134 from the drawing assessment features 226 and/or metadata features 212 and employ the features in a supervised or unsupervised machine learning operation to generate an estimated value (e.g., score 138) for the likelihood of the presence or non-presence of the cognitive disease, condition, or an indicator of either. The likelihood may be evaluated against a pre-defined threshold value to provide an indication of the presence or non-presence of the cognitive disease, condition, or an indicator of either. The output of the analysis system 106 can be provided to the client 112. In some embodiments, the analysis system 106 is configured to provide the output to a different portal to be viewed by a clinician and/or user. The score 138 can be used by a healthcare provider, a test evaluator, or a user to assist in a diagnosis of a cognitive disease or condition.

In some embodiments, the one or more machine learning models 134 are used to generate a score for a cognitive level function that is associated with cognitive performance relating to memory, critical thinking, focus, etc. The generated score 138 can be used by a healthcare provider or a test evaluator to quantify a cognitive function.

In some embodiments, the one or more machine learning models 134 are used to generate a score 138 for a cognitive level function that is associated with cognitive performance relating to a training or education-related activity. The generated cognitive level function can, for example, be evaluated over time to determine change in assessed cognitive level function.

Machine Learning. In addition to the machine learning features described above, the analysis system 106 can be implemented using one or more artificial intelligence and machine learning operations performed by the one or more machine learning models 134. The term “artificial intelligence” can include any technique that enables one or more computing devices or comping systems (i.e., a machine) to mimic human intelligence. Artificial intelligence (AI) includes, but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the model discovers a pattern (e.g., structure, distribution, etc.) within an unlabeled or labeled data set. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is a supervised classification model that classifies new data points based on similarity measures (e.g., distance functions). k-NN classifier is a non-parametric algorithm, i.e., it does not make strong assumptions about the function mapping input to output and therefore has flexibility to find a function that best fits the data. The k-NN classifiers are trained with a data set (also referred to herein as a “dataset”) by learning associations between all samples and classification labels in the training dataset. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Example #2

FIG. 1B shows an example system 100b configured to detect cognitive impairment (e.g., early cognitive impairment) or assess cognitive function using artificial intelligence analysis in accordance with an illustrative embodiment. In FIG. 1B, like numerals are used to show like parts, where reference is made to the description of these common parts above in conjunction with FIG. 1A.

In FIG. 1B, the system 100b includes the cognitive test portal 102 and an integrated test assessment server 104 and analysis system 106. The analysis system 106 includes artificial intelligence analysis and/or machine learning analysis configured to detect cognitive impairment (e.g., early cognitive impairment) or assess cognitive function. In FIG. 1B, the analysis system 106 includes a metadata extraction module 130 (shown as “Metadata Features”) that is configured to determine the score of a given test response, the time spent on each question, as well as frequency and/or count of the user changing the test questions (e.g., timestamps for when the user clicked “next page” or “previous page”), or other behavioral metadata discussed above. The analysis system 106 additionally includes a drawing component analysis module 132 (shown as a “Drawing Features” module) that is configured to identify, for each instance in the time and position log, an entry position and entry time for a given stroke and an exit position and an exit time for the given stroke. The drawing component analysis module 132 can extract, measure, or determine the drawn metadata measures associated with the speed, accuracy, and consistency of the user's response in which the measure is calculated from the time and position log or recorded entry positions, entry times, exit positions, and exit times for each stroke. Though shown as separate modules, the metadata extraction module 130 and drawing component analysis module 132 may be implemented in a single module. Example drawing features are provided above in Table 1.

The analysis system 106, in some embodiments, is configured to generate or train one or more machine learning models 134 from the drawing component features and/or metadata features and employ the features in a supervised or unsupervised machine learning operation to generate an estimated value (e.g., score 138) for the likelihood of the presence or non-presence of the cognitive disease, condition, or an indicator of either. The likelihood may be evaluated against a pre-defined threshold value to provide an indication of the presence or non-presence of the cognitive disease, condition, or an indicator of either.

As shown in FIG. 1B, the output of the analysis system 106 can be provided to the test assessment server 104 to provide a unified output to the client 112.

The score 138 can be used by a healthcare provider, a test evaluator, or a user to assist in a diagnosis of a cognitive disease or condition. In some embodiments, the machine learning operation is used to generate a score 138 for a cognitive level function that is associated with cognitive performance relating to memory, critical thinking, focus, etc. The generated score 138 can be used by a healthcare provider or a test evaluator to quantify a cognitive function. In some embodiments, the machine learning operation is used to generate a score 138 for a cognitive level function that is associated with cognitive performance relating to a training or education-related activity. The generated cognitive level function can, for example, be evaluated over time to determine change in assessed cognitive level function, e.g., as described in relation to FIG. 1A.

### Example #3

FIG. 1C shows an example system 100c configured to detect cognitive impairment (e.g., early cognitive impairment) or assess cognitive function using artificial intelligence analysis in accordance with an illustrative embodiment. In FIG. 1C, like numerals are used to show like parts, where reference is made to the description of these common parts above in conjunction with FIG. 1A.

In FIG. 1C, the analysis system, e.g., of FIG. 1A or 1B, is configured with to perform artificial intelligence analysis and/or machine learning analysis configured to detect cognitive impairment (e.g., early cognitive impairment) or assess cognitive function. In FIG. 1C, the analysis system 106 includes a metadata extraction module 130 (shown as “Metadata Features”) that is configured to determine the score of a given test response, the time spent on each question, as well as frequency and/or count of the user changing the test questions (e.g., timestamps for when the user clicked “next page” or “previous page”), or other behavioral metadata discussed above. The analysis system 106 additionally includes a drawing component analysis module 132 (shown as a “Drawing Features” module) that is configured to identify, for each instance in the time and position log, an entry position and entry time for a given stroke and an exit position and an exit time for the given stroke. The drawing component analysis module 132 can extract, measure, or determine the metadata measures associated with the speed, accuracy, and consistency of the user's response in which the measure is calculated from the time and position log or recorded entry positions, entry times, exit positions, and exit times for the given stroke. Though shown as separate modules, the metadata extraction module 130 and drawing component analysis module 132 may be implemented in a single module. Example drawing features are provided above in Table 1.

In addition, in FIG. 1C, the analysis system 106 is further configured to employ patient-associated medical data from an electronic medical record (shown as “EMR Database 140”). In some implementations, the behavioral analysis system 136 may evaluate fields in the EMR database 140 to generate or extract one or more features 142 that are useful for determining the likelihood of the presence or non-presence of the cognitive disease, condition, or an indicator of either.

The analysis system 106, in some embodiments, is configured to generate one or more machine learning models 134 from the cognitive test scoring and results data, drawing component features, metadata features, and EMR features 142 and employ the features in a supervised or unsupervised machine learning operation to generate estimated value (e.g., score 138) for the likelihood of the presence or non-presence of the cognitive disease, condition, or an indicator of either. The likelihood may be evaluated against a pre-defined threshold value to provide an indication of the presence or non-presence of the cognitive disease, condition, or an indicator of either. The output of the analysis system 106 can be provided to the test assessment server 104 to provide a unified output to the client 112.

The score 138 can be used by a healthcare provider, a test evaluator, or a user to assist in a diagnosis of a cognitive disease or condition. In some embodiments, the machine learning operation is used to generate a score 138 for a cognitive level function that is associated with cognitive performance relating to memory, critical thinking, focus, etc. The generated score 138 can be used by a healthcare provider or a test evaluator to quantify a cognitive function. In some embodiments, the machine learning operation is used to generate a score 138 for a cognitive level function that is associated with cognitive performance relating to a training or education-related activity. The generated cognitive level function can, for example, be evaluated over time to determine change in assessed cognitive level function.

The EMR database 140 includes patient-specific medical records, including patient medical history, family history as well as past and current medical-related information and treatment history. An electronic medical record refers to the systemic collection of patient and population electronically-stored health information in a digital format. These records can be shared across different health care settings. Records are shared through network-connected, enterprise-wide information systems or other information networks and exchanges.

Example of EMR data includes patient demographic information, progress notes, vital signs, medical histories, diagnoses, medications, immunization dates, allergies, radiology images, and lab and test results.

In some embodiments, EMR data may include administrative and billing data.

In some embodiments, for cognitive impairment assessments, the analysis system 106 and/or the behavioral analysis system 136 may extract demographic information, progress notes, vital signs, medical histories, diagnoses, medications, immunization dates, allergies, family history of cognitive conditions, laboratory evaluations (e.g. TSH, B12, metabolic tests, etc. . . . ) relating to cognitive function, and radiology images including specific areas of focal atrophy or focal radiographically seen pathology (e.g. white matter disease, vascular disease, masses, etc.) relating to cognitive function.

Example Computing System. The analysis system as well as various computing devices of FIGS. 1A, 1B, and/or 1C may be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

The various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### Experimental Results and Example

A study was conducted to develop and evaluate test machine learning methods to detect cognitive impairment using results from an electronic cognitive test and to evaluate behavioral metadata with the electronic cognitive test as novel biomarkers. The study employed an electronic cognitive test, “eSAGE.” The study evaluated the different behavioral patterns (such as spending more time on questions and changing answers often) exhibited by those with a diagnosis of normal cognition, mild cognitive impairment, or dementia to expand the toolset available to detect cognitive disorders, e.g., contributing to earlier detection of Alzheimer's disease.

In this research study, machine learning techniques were investigated to predict an individual's cognitive impairment (CI) stage from eSAGE scores and metadata, and to evaluate behavioral features (metadata) during eSAGE tests (e.g., time spent on each question, drawing qualities) as novel biomarkers. The hypothesis was that cognitively impaired patients show different behavioral patterns (e.g., spend more time on questions, draw shorter lines per stroke) than those of normal cognition (NC). By leveraging machine learning techniques, we also aimed to expand the toolset available to accurately detect cognitive disorders, contributing to earlier detection of dementia conditions like AD. The wide accessibility and ease-of-use of such tools (e.g., eSAGE) will enable early and easy assessment and hopefully early management and treatment of cognitive impairment. Also, by demonstrating the benefits of applying machine learning techniques to eSAGE data, it opens up the possibility of applying machine learning with other cognitive test data to further improve the tools available to be used as novel biomarkers to identify AD.

Methodology. The study obtained the results and behavioral data from BrainTest® for 66 subjects, each with a diagnosis of normal cognition, mild cognitive impairment (MCI), or dementia. Table 2 summarizes the information about the available data. The eSAGE test includes questions asking for information about the test taker (such as date of birth and health history), as well as calculation problems, simple puzzles, and drawing problems. The data provided from the test included the response, the score of the response, and the time spent on each question, in addition to the timestamps for when they clicked “next page” or “previous page.” For the drawing problems, additional information was available, such as timestamps and the position they were drawing in at that timestamp.

Each eSAGE question was scored and behavioral features (metadata) such as the time spent on each test page, drawing speed and average stroke length were extracted for each subject. Logistic regression and gradient boosting models were trained using these features to detect cognitive impairment. Performance was evaluated using five-fold cross validation, with accuracy, precision, recall, F1 score, and ROC AUC score as evaluation metrics.

TABLE 2

| Diagnosis | Normal Cognition | MCI | Dementia |
|---|---|---|---|
| Count | 21 subjects | 24 subjects | 21 subjects |

The self-administered eSAGE test measures cognitive function in the domains of orientation (date: 4 points), language (picture naming: 2 points and verbal fluency: 2 points), memory (2 points), executive function (modified Trails B: 2 points and problem solving task: 2 points), abstraction (2 points), calculations (2 points), and visuospatial abilities (copying 3-dimensional constructions: 2 points and clock drawing: 2 points). Non-scored items include demographic information (birthdate, educational achievement, ethnicity, and sex), and questions regarding the individual's past history of strokes and head trauma, family history of cognitive impairment, and current symptoms of memory, balance, mood, personality changes, and impairments of activities of daily living.

No training is required for the administration of the test and assistance is not allowed. Clocks and calendars were not allowed in the testing rooms. Answers need not be spelled correctly. There is no time limit to complete the test. The subjects used their fingers to draw or type to complete the eSAGE questions on the tablet. The eSAGE design allowed for the subjects to write on the tablet as a scratch pad to aid in the completion of the calculations section. The subjects had the ability to delete and retype, or return and alter any answer to any question at any time. For those visuospatial and executive functioning questions requiring them to draw with their finger, there was the ability to erase their entire answer and redraw or redo their answer. If more than one response was provided for a question, the best response was scored.

Feature Engineering. The study developed a set of behavioral features associated with the hand drawing associated features. In total, 103 features were calculated from text-only questions and from combined text and drawing questions.

Three types of behavioral features were generated. First, the time spent on each question was extracted from the logs provided by the BrainTest® system. The motivation was that cognitively impaired people might spend longer on each question than those with normal cognition (e.g., due to difficulty understanding the questions).

Second, the number of times the test taker went back 1 page, 2 pages, 3 pages, and so on were calculated from the logs as well. The intent was that cognitively impaired people might tend to backtrack a lot while taking the test.

Third, another category of features was calculated from the drawing problems. The aim was to quantify the quality of the drawings, as cognitively impaired individuals may draw worse-than-average drawings. For each drawing problem, the BrainTest® system administered the eSAGE test provided a log with the timestamp and the corresponding coordinate at which the patient was drawing, as well as information about when the patient lifted their finger to start a new stroke. From these logs, the total time taken, the total length of the drawing, and the number of strokes were calculated, along with other metrics such as average length and average speed per stroke. Also, the convex hull area and perimeters were used as features.

Knowing how a patient copied a figure can indicate if they employed a piecemeal approach or performed the task by starting with the outline initially and then filling in internal features. Average stroke length can provide a measure of their approach. The time it takes to complete a task, the average stroke speed, whether their drawing was much smaller or larger than the original, and the number of times they tried to draw the construction, all give valuable information that relate to their cognitive functioning. In addition, the straightness of lines in drawings was quantified and used as a feature.

FIG. 3B shows an example of a stroke (shown in black) starting from point (a, b) and ending at point (c, d). Qualitatively, a stroke is more “straight” when the length of the stroke is close to the distance between the endpoints of the stroke. Straightness can thus be defined as the ratio of the distance between the endpoints to the length of the stroke. This straightness metric was calculated for each stroke and averaged for all of the strokes in the drawing to compute the average straightness per stroke.

Because some patients would draw all four sides of a rectangle in a single stroke, the metric can yield a low straightness value because the start point and endpoint of the stroke were very close together. This problem was most evident in the drawing problem, where patients were tasked with copying a picture of a cube. The study split a single-stroke rectangle at its corners and treated it as four separate strokes. The boundary box of the drawn rectangle was calculated, and the point that was closest to each corner of the boundary box was called the corner of the drawing.

The score of each response to each question was used as feature as well. Scores of those with MCI or dementia may be lower than those with normal cognition.

Models. Using these features, logistic regression models and gradient boosting classifiers were trained to predict the level of cognition: either normal cognition, mild cognitive impairment (MCI), or dementia. For this task, the scikit-learn (Pedregosa, et al., 2011) implementations of the models were used.

Logistic regression models are binary classifiers that output the probability of the input data being part of the “positive” class (as opposed to the “negative” class). The goal of logistic regression is to determine the weights w and bias c in Equation 1, where f is the output of the model, and x is a vector of features of the sample that is being classified.

$$\hat{y} ( w , x ) = \frac{1}{1 + exp ( - ( w \cdot x + c ) )} \qquad ( Eq . 1 )$$

The objective is to minimize the cost function as shown below in Equation 2 where xᵢ is the i-th training sample and yᵢ is the expected output of the i-th training sample. Elastic-Net regularization can be added as shown below in Equation 3, where p is the strength of 11 regularization vs. 12 regularization and C is the inverse of the strength of regularization. Regularization penalizes larger weight parameters, so it prevents overfitting of the training data.

$${min}_{w , c} {\sum }_{i = 1}^{n} log ( exp ( - {γ}_{i} ( w \cdot {x}_{i} + c ) ) + 1 ) \qquad ( Eq . 2 )$$

$${min}_{w , C} \frac{1 - ρ}{2} {w}^{T} w + ρ {\vert w \vert}_{1} + C {\sum }_{i = 1}^{n} log ( exp ( - {y}_{i} ( w \cdot {x}_{i} + c ) ) + 1 ) \qquad ( Eq . 3 )$$

Gradient boosting trees are an ensemble of decision trees. The training begins with a constant model (which classifies all inputs the same) and to iteratively add decision trees to the model. These new decision trees are fit to the residual error in the model at that step, in order to learn parts of the problem that the model hasn't figured out yet. Algorithm 1 shows the algorithm to train gradient boosting trees (Friedman, 1999). The final model is F_{M}(x), and L(yᵢ, ŷᵢ) is the loss function. The default loss function in scikit-learn is negative binomial log-likelihood loss.

Algorithm 1: Gradient_Boost (Friedman, 1999)

| | |
|---|---|
| 1 | ${F}_{0} ( x ) = arg {min}_{ρ} {\sum }_{i = 1}^{N} L ( {y}_{i} , ρ )$ |
| 2 | for m = 1 to M do |
| 3 | $\tilde{{y}_{ι}} = - {[ \frac{\partial L ( {y}_{i} , F ( {x}_{i} ) )}{\partial F ( {x}_{i} )} ]}_{F ( x ) = {F}_{m - 1} ( x )}$ |
| 4 | Fit a decision tree h(x) to data, using \tilde{y} as targets |
| 5 | ${ρ}_{m} = arg {min}_{ρ} {\sum }_{i = 1}^{N} L ( {y}_{i} , {F}_{m - 1} ( {x}_{i} ) + ρ h ( {x}_{i} ) )$ |
| 6 | Fₘ(x) = Fₘ₋₁(x) + ρₘh(x) |
| 7 | end for |

Experimental Protocol. The models were trained as binary classifiers. Since this was a multiclass classification problem, multiple models with different classification goals were trained (listed positive vs. negative case): cognitive impairment vs. normal cognition, MCI vs. normal cognition, dementia vs. normal cognition, dementia vs. MCI, dementia vs. normal cognition, or MCI, MCI vs. normal cognition or dementia. The classification goals were organized so that each negative case was closer to normal cognition than the corresponding positive case. The first three classification goals were arguably the most important, as they could be used to detect cognitive issues. The other three classification goals were included for completeness.

All of the features used were normalized to be between “0” and “1” using min-max normalization. The features were split into two categories. score (including only the scores) and behavioral (including all of the features other than score). Then, models for the six classification goals were trained using three different sets of features (score only, behavioral only, and all features), as well as different parameter settings (Table 1). The objective was to find the best-performing models for each of the classification goals and to obtain the most important features.

To evaluate the performance of each model, five-fold cross-validation was used. Cross-validation was chosen because of the limited dataset available to test and train on. In five-fold cross-validation, the datasets were randomly shuffled and split into five folds. One fold was held out as the test dataset, and a model was trained using the other four folds are used as the training dataset. Then, the model was evaluated using the test dataset, using metrics such as accuracy. This process was repeated four more times, with each fold having one round as the test dataset and four rounds as part of the training dataset. Finally, the evaluation metrics were averaged to give an overall evaluation result.

Evaluation Metrics. For each classification goal, multiple models with different parameters (such as regularization weight and learning rate) were trained and evaluated. Specifically, for the performance metrics, the average accuracy, precision, recall, F1 score, and ROC AUC score were calculated for each of the five folds. For all of these performance metrics, a higher value is indicative of better performance. The equations for accuracy, precision, recall, and F1 score are Equation 4 through Equation 7 below. In each equation, TP, FP, TN, and FN are the numbers of true positives, false positives, true negatives, and false negatives, respectively. Finally, the ROC AUC score is the area under the receiver operating characteristic curve, serving as another measurement of overall performance.

$$accuracy = \frac{T P + T N}{T P + F P + T N + F N} \qquad ( Eq . 4 )$$

$$precision = \frac{T P}{T P + F P} \qquad ( Eq . 5 )$$

$$recall = \frac{T P}{T P + F N} \qquad ( Eq . 6 )$$

$$F 1 score = \frac{2 \cdot precision \cdot recall}{precision + recall} \qquad ( Eq . 7 )$$

After training all of the models, the study chose the best model for each classification goal based on the performance metrics. Within each model, the features with large corresponding feature weights (absolute value) were considered important, as they have larger impacts on the classification output than features were smaller weights. The five most important features were observed for each of the models with the best performance.

### Results

Overall Performance. Table 3 shows the overall performance for the five classification tasks. For each classification task, the best performance in terms of AUC was selected, and its corresponding accuracy (“acc”), precision (“prec”), recall (“rec”), and F1 values were presented as well. Overall, Table 3 demonstrates that behavioral information in addition to eSAGE scoring information is particularly useful to help detect mild cognitive impairment and dementia from normal cognition.

TABLE 3

| Tasks | Classes | Features | Model | acc | prec | rec | F1 | AUC. |
|---|---|---|---|---|---|---|---|---|
| Task 1 | CI vs. NC | F-b | LR (l₁, l₂) | 0.6821 | 0.7121 | 0.9111 | 0.7958 | 0.7731 |
| | | F-s | LR (l₁, l₂) | 0.7528 | 0.8170 | 0.8661 | 0.8279 | 0.9177 |
| | | F-bs | LR (l₁, l₂) | 0.7869 | 0.8345 | 0.8911 | 0.8493 | 0.9288 |
| Task 2 | MCI vs. NC | F-b | LR (l₁, l₂) | 0.6622 | 0.6933 | 0.7600 | 0.7055 | 0.7945 |
| | | F-s | LR (l₁, l₂) | 0.6606 | 0.7676 | 0.6300 | 0.6587 | 0.8640 |
| | | F-bs | LR (l₁, l₂) | 0.6606 | 0.7343 | 0.6700 | 0.6628 | 0.8870 |
| Task 3 | DM vs. NC | F-b | GBC | 0.7350 | 0.7467 | 0.7100 | 0.7219 | 0.7520 |
| | | F-s | LR (l₂) | 0.9600 | 1.0000 | 0.9200 | 0.9500 | 0.9840 |
| | | F-bs | LR (l₂) | 0.9600 | 1.0000 | 0.9200 | 0.9500 | 0.9920 |
| Task 4 | DM vs. MCI | F-b | GBC | 0.6217 | 0.5600 | 0.5500 | 0.5333 | 0.6800 |
| | | F-s | LR (l₁, l₂) | 0.7589 | 0.8033 | 0.7400 | 0.7100 | 0.9125 |
| | | F-bs | LR (l₁, l₂) | 0.7589 | 0.8033 | 0.7400 | 0.7100 | 0.9125 |
| Task 5 | DM vs. nDM | F-b | LR (l₂) | 0.7549 | 0.7000 | 0.5200 | 0.5633 | 0.7180 |
| | | F-s | LR (l₂) | 0.8210 | 0.8243 | 0.6900 | 0.6732 | 0.9457 |
| | | F-bs | LR (l₁) | 0.8672 | 0.9100 | 0.6900 | 0.7278 | 0.9426 |

Logistic regression with feature selection achieved an AUC of 92.88%, a recall of 89.11% and an F1 of 84.93% using both behavioral and scoring features together to classify cognitive impairment vs. normal cognition. This was better than logistic regression using only scoring features, which achieved an AUC of 91.77%, a recall of 86.61%, and an F1 of 82.73%, demonstrating the strong potential of using scores and metadata together in detecting cognitive impairment. Logistic regression using scores and metadata also achieved an AUC of 88.70% in detecting mild cognitive impairment from normal cognition, and an AUC of 99.20% in detecting dementia from normal cognition. Average stroke length was particularly useful for prediction and when combined with 4 other scoring features, logistic regression achieved an even better AUC of 94.06% in detecting cognitive impairment.

For each classification task, the best performance in terms of AUC was selected, and its corresponding acc., prec., rec. and F1 values were presented as well. To classify CI vs. NC in Task 1, F-b only with LR (with elesticnet regularization as shown later) can achieve an AUC value at 0.7731, well above 0.5 corresponding to random guessing, indicating the utility of behavioral information collected during eSAGE testing for detecting CI. This also conforms to the research in the literature that subjects' behaviors are indicative of their cognition status. Meanwhile, F-s only with LR can achieve a much higher AUC value at 0.9177. This confirms the previous conclusion on the robustness and effectiveness of eSAGE in detecting CI.

When both F-b and F-s were used together in F-bs, AUC value was further improved to 0.9288, that is, a 1.2% improvement of the AUC of F-s alone. This indicates that behavioral data can provide complementary information that was not captured by eSAGE scores to further improve CI detection, further verifying the utility of behavioral information collected during eSAGE testing. Particularly, in correspondence to the best AUC values, F-bs achieved a rec value 0.8911, that is, a 2.9% improvement from the rec of F-s, demonstrating that behavioral information and scoring information together could detect true CI more effectively than scoring information alone.

In addition, F-bs achieved better acc and F1 values than F-s. Notably, F-b was able to achieve a rec value at 0.9111 but a low prec at 0.7121, showing that F-b tends to predict more towards CI; F-s was conservative compared to F-b in predicting CI, and thus had a lower rec value at 0.8661 but a higher prec. at 0.8170. F-bs mitigates the issues of F-b and F-s, and thus had both reasonable prec. and rec. values.

Results for classifying MCI vs. NC share similar trends as those discussed above. F-bs achieved the best AUC 0.8870 with a 2.7% improvement from that of F-s alone. Comparing the two tasks Task 1 (CI vs. NC) and Task 2 (MCI vs. NC), where CI includes both MCI and DM, Task 2 was more difficult (with lower AUC). This could be because that DM has more distinguishable features that better separate DM subjects from those of normal cognition. This was further demonstrated by the performance of Task 3 (DM vs. NC), which had the best AUC value at 0.9920, much higher than those in Task 1 and 2. However, it was noted that for Task 3, F-s and F-bs performed almost the same with only a 0.8% improvement from F-bs on AUC, and F-b contributed very minimally in F-bs. This further validated that F-s in eSAGE is very effective in detecting DM from NC.

The same observation was true for Task 4 to classify DM vs. MCI, in which F-bs and F-s have identical performance. However, Task 4 was more difficult than Task 3 as DM and MCI, both belonging to cognitive impairment, share more similar traits compared to those of DM vs. NC. This is further validated by the much lower AUC 0.9125 compared to that in Task 3 (0.9920). For Task 5 to classify DM vs nDM, again, both F-bs and F-s resulted in very similar AUC (0.9426 vs. 0.9457), though F-bs had better performance in terms of acc., prec., and F1. Overall, Table 3 demonstrates that behavioral information in addition to eSAGE scoring information is particularly useful to help detect mild cognitive impairment and dementia from normal cognition.

Important Features. Table 4 shows the most important features (the highest weighted ones) when both behavioral features and scoring information are used. Table 5 shows the most important features (the highest weighted ones) when only behavioral features are used.

TABLE 4

| Task | Classes | Model | Important Features | Weight |
|---|---|---|---|---|
| Task 1 | CI vs. NC | LR (l₁, l₂) | Modified Trails Score | −0.9131 |
| | | | Memory Question Score | −0.8208 |
| | | | Verbal Fluency Score | −0.7789 |
| | | | Date Question Score | −0.4176 |
| | | | Modified Trails Average Stroke Length | −0.2095 |
| Task 2 | MCI vs. NC | LR (l₁, l₂) | Modified Trails Score | −0.7475 |
| | | | Verbal Fluency Score | −0.5374 |
| | | | Memory Question Score | −0.3952 |
| | | | Modified Trails Average Stroke Length | −0.1199 |
| | | | Picture Naming Question 2 Score | −0.0885 |
| Task 3 | DM vs. NC | LR (l₂) | Memory Question Score | −0.7777 |
| | | | Modified Trails Score | −0.5869 |
| | | | Date Question Score | −0.5387 |
| | | | Verbal Fluency Question Score | −0.4738 |
| | | | Orientation Questions Score | −0.4214 |
| Task 4 | DM vs. MCI | LR (l₁, l₂) | Calculation Question 2 Score | −1.1915 |
| | | | Date Question Score | −0.5536 |
| | | | Clock Drawing Score | −0.4937 |
| | | | Similarity Question Score | −0.4133 |
| | | | Memory Question Score | −0.2582 |
| Task 5 | DM vs. nDM | LR (l₁) | Calculation Question 2 Score | −1.1032 |
| | | | Memory Question Score | −1.0597 |
| | | | Date Question Score | −0.9930 |
| | | | Clock Drawing Score | −0.5646 |
| | | | Similarity Question Score | −0.2120 |

TABLE 5

| Task | Classes | Model | Important Features | Weight |
|---|---|---|---|---|
| Task 1 | CI vs. NC | LR (l₁, l₂) | Modified Trails Average Stroke Length | −0.9167 |
| | | | Date Question Time | 0.6696 |
| | | | Orientation Questions Time | 0.6112 |
| | | | Language Questions Time | 0.4716 |
| | | | Verbal Fluency Question Time | 0.4326 |
| Task 2 | MCI vs. NC | LR (l₁, l₂) | Modified Trails Average Stroke Length | −0.4494 |
| | | | Date Question Time | 0.3637 |
| | | | Language Questions Time | 0.3120 |
| | | | Verbal Fluency Question Time | 0.2960 |
| | | | Picture Naming Question 2 Time | 0.2570 |
| Task 3 | DM vs. NC | GBC | Modified Trails Time | 0.3924 |
| | | | Sad, Depressed Question Time | 0.0749 |
| | | | Clock Drawing Time | 0.0583 |
| | | | Language Questions Time | 0.0565 |
| | | | Modified Trails Stroke Straightness | 0.0444 |
| Task 4 | DM vs. MCI | GBC | Education Question Time | 0.1086 |
| | | | Clock Drawing Average Stroke Speed | 0.1009 |
| | | | Modified Trails Stroke Straightness | 0.1003 |
| | | | Cognitive Functions Question Time | 0.0784 |
| | | | Month Question Time | 0.0620 |
| Task 5 | DM vs. nDM | LR (l₂) | Modified Trails Time | 0.1166 |
| | | | Clock Drawing Time | 0.1031 |
| | | | Problem Solving Drawing Total Length | 0.0738 |
| | | | Month Question Time | 0.0522 |
| | | | Modified Trails Stroke Straightness | 0.0481 |

For logistic regression models (LR), a positive/negative weight indicated that a higher/lower feature value contributed to a positive classification. Since elasticnet regularizer (i.e., }₁ and }₂ regularization) was applied in LR, which utilizes }ₜ-norm regularization to performance automatic feature selection, the important features were also among the selected features.

Table 4 shows that for Task 1 (CI vs. NC), the top-5 most important features included four scores from eSAGE and a behavioral feature. Among the four eSAGE scores, Modified Trails Score measures the score on the modified trails problem, where correctly connecting the circles in order leads to a higher score. The Memory Question Score measures the subject's memory by asking them to recall a phrase at the end of the test. The Verbal Fluency Score measures the subject's ability to write down 12 distinct items of a particular class (e.g., animals). The Date Question Score measures the subject's knowledge of the current date. Modified Trails Average Stroke Length measures the average length per stroke on the modified trails B task, and the negative weight indicated that a longer average length tended toward classification as normal cognition.

Task 2 (MCI vs. NC) shared the top-3 most important features as Task 1, all from eSAGE. Modified Trails Average Stroke Length became the 4-th important feature in Task 4, more important than in Task 1. The Picture Naming Question emerged as the 5-th important feature, which measures the subject's writing ability by presenting a picture (e.g., a volcano erupting) and asking the subject to describe it. For Task 3, 4 and 5, their top-5 most important features were all from eSAGE.

Table 5 presents the top-5 important features when only behavioral features were used. Although behavioral features alone did not enable competitive performance compared to both behavioral and scoring features together, behavioral features that were important could still shed lights on further development utilizing them. Table 6 shows that for Task 1 (CI vs. NC), in addition to Modified Trails Average Stroke Length, timing features demonstrate positive relations to CI, that is, if a subject spends a longer time on a question, he/she is more likely to be classified toward CI.

Parameter Study. The parameters for each model are shown in Table 6. For logistic regression, Elasticnet was used for all models for the first two classification goals. This penalty was mostly used alongside a strong regularization strength (C>=0.5), which indicated that avoiding overfitting led to better performance. For gradient boosting trees, a learning rate of 0.1 or 0.01 tended to perform the best. The minimum sample split for dementia vs. normal cognition, dementia vs. MCI, and dementia vs. normal cognition or MCI was 4, which indicated that it was better to keep the leaf nodes in the tree larger in order to avoid overfitting to the noise in the training set.

Options and ranges for the parameters includes regularization: l₁, l₂, l₁ and l₂; C (inverse of regularization strength): 0.5, 1, 2; l₁ Ratio: 0.25, 0.5, 0.75; Learning Rate: 0.001, 0.01, 0.1; Max Depth (maximum depth of each decision tree): 3, 4, 5; Min Split (minimum number of samples needed to split an internal node): 2, 3, 4.

TABLE 6

| | | | | | | | Learning | Max | Min |
|---|---|---|---|---|---|---|---|---|---|
| Tasks | Classes | Features | Model | Regularization | C | l₁ Radio | Rate | Depth | Split |
| Task 1 | CI vs. NC | F-b | LR | l₁, l₂ | 1.0 | 0.50 | — | — | — |
| | | F-s | LR | l₁, l₂ | 0.5 | 0.50 | — | — | — |
| | | F-bs | LR | l₁, l₂ | 0.5 | 0.75 | — | — | — |
| | | top-5 | LB | l₂ | 2.0 | — | — | — | |
| Task 2 | MCI vs. NC | F-b | LR | l₁, l₂ | 0.5 | 0.25 | — | — | — |
| | | F-s | LR | l₁, l₂ | 0.5 | 0.75 | — | — | — |
| | | F-bs | LR | l₁, l₂ | 0.5 | 0.75 | — | — | — |
| | | top-5 | LR | l₂ | 0.5 | — | — | — | |
| Task 3 | DM vs. NC | F-b | GBC | — | — | — | 0.01 | 5 | 4 |
| | | F-s | LR | l₂ | 2.0 | — | — | — | — |
| | | F-bs | LR | l₂ | 0.5 | — | — | — | — |
| | | top-5 | LR | l₂ | 2.0 | — | — | — | |
| Task 4 | DM vs. MCI | F-b | GBC | — | — | — | 0.10 | 4 | 4 |
| | | F-s | LR | l₁, l₂ | 0.5 | 0.75 | — | — | — |
| | | F-bs | LR | l₁, l₂ | 0.5 | 0.75 | — | — | — |
| | | top-5 | LR | l₂ | 2.0 | — | — | — | |
| Task S | DM vs. nDM | F-b | LR | l₂ | 2.0 | — | — | — | — |
| | | F-s | LR | l₂ | 0.5 | — | — | — | — |
| | | F-bs | LR | l₁ | 0.5 | — | — | — | — |
| | | top-5 | LR | l₂ | 2.0 | — | — | — | |

### Discussion

Alzheimer's disease is a neurodegenerative disease that affects many people worldwide. In 2019, an estimated 5.8 million Americans had Alzheimer's dementia, and the number is expected to increase to 13.8 million by mid-century (Alzheimer's Association, 2019). It is a progressive disease that causes irreversible brain damage (Alzheimer's Association, 2019; Evans, Funkenstein, & Albert, 1989; Grossberg, 2003). The time between symptoms arising and death can span 8 to 10 years (Grossberg, 2003), so an accurate early diagnosis of the disease is desirable because it allows for measures to be taken to prevent the worsening of the symptoms.

Many cognitive screening tests are available, with one example being Self-administered Gerocognitive Examination (SAGE) (Scharre, et al., 2010). It was designed to be self-administered, not requiring any special equipment or personnel time. A digital version of SAGE (called eSAGE) that could be administered on a tablet was later created (Scharre, Chang, Nagaraja, Vrettos, & Bornstein, 2017). It was implemented as an app in BrainTest®, with sample screenshots in Table 3B. While the eSAGE exam provides diagnostic results of its own, there is a potential to apply machine learning methods to the responses to obtain better diagnostic results. This approach of applying machine learning techniques to neuropsychological tests has been tried before, such as with the Digital Clock Drawing Test (Binaco, et al., 2020) and the Alzheimer's disease assessment scale cognitive test (Lahmiri & Shmuel, 2019).

Clinically, the need to differentiate those with normal cognition from those that are impaired (Task 1) is useful to making an accurate and specific diagnosis. In neurodegenerative disorders like AD, with the advent of disease modifying agents, the ability to identify MCI from normal cognition (Task 2) is necessary if we are to start treatments earlier. The ability to differentiate dementia stages from MCI or non-dementia stages (Task 4 and 5) is clinically important for treatment and management considerations. Task 3 is the least useful pairing since it is fairly easy to differentiate between dementia and normal groups clinically.

Table 3 provides the summary data from the five classification tasks. In addition to the acc., prec., rec., and F1 metrics, we have listed the AUC from ROC analyses. Particularly in looking at the AUC for the score features of eSAGE (F-s) and for the score and behavioral features combined of eSAGE (F-bs), we find that the AUC show values of 0.86 and above. AUC numbers above 0.8 are generally considered very good for clinical use especially in differentiating NC from MCI and MCI from DM (Tasks 1 and 2).

For Task 1 (NC vs CI), Table 5 listed the top 5 most impactful features when both the score and metadata of eSAGE are used. The modified Trails B task average stroke length was the only non-score, behavioral feature that made the list. This task involves the individual to draw a line connecting numbers and letters in alternating fashion in ascending order. Generally, if you have slower cognitive processing speed and it takes you longer to decide which is the next circle to connect in sequence, the more likely you will interrupt your connections of the circles. It was interesting to note that this behavioral feature was also in the top 5 most impactful features for Task 2 (NC vs MCI) and that the score for the modified Trails B task was the most impactful feature for both Task 1 and 2. It is well appreciated that Trails B is a very sensitive executive measure. Our analyses show that it's use in eSAGE contributes significantly to the helping differentiating normal individuals from MCI and CI. By adding the behavioral feature of average stroke length, the impact of using the modified Trails B is enhanced.

In Task 1, the cognitively impaired group includes those with dementia. Disorientation to date is very common in those with dementia more than those with MCI and so it makes sense that we find disorientation to date as a more important feature in Table 4 when comparing NC and CI (Task 1) than when comparing NC and MCI alone (Task 2). Forgetting names of objects is an early language impairment seen in MCI patients and we see it is more impactful than orientation to date for Task 2.

Finally in Table 4, we note that for Task 3, 4 and 5, the top-5 most impactful features were all from just the scores of certain questions from eSAGE. No behavioral features made the list. For differentiating DM from MCI (Task 4) or nDM (Task 5), the top 5 features were the same but just with the memory score showing a different order of impact. The memory score moved higher in Task 5 as this task includes normal individuals in the comparison and so a lower memory score would suggest more likelihood of dementia. As would be expected, the memory score moves to the most impactful in Task 3 when comparing only dementia and normal cognition folks. Consistently, in Task 3 (normal versus dementia individuals), we see similar question scores showing high impacts in Table 4 that we see in Task 1 (normal from MCI individuals).

In Table 5, we found that the how long it takes a person to finish a question (timing) are also impactful behavioral features. Knowing the correct date (score) and the ability to more quickly come up with the date, month, and year answers (timing) are consistent with not being diagnosed with cognitive impairment. While processing speed slows with aging, it you are aware of the exact date, you will write it down on the test fairly quickly. However if you are coming up with possible date answers and then rejecting them and then deciding on another date the processing speed and timing are longer. This is also true for naming pictures and coming up with nouns that make up a category (e.g. animals). The individual has to think of a word and then either reject and think of some more possibilities, or accept the word. It makes sense that the time to do the verbal fluency task shows a high impact; likely due to the number of answers (12) that are required. Those individuals with MCI or DM often have impairments in frontal or executive circuits. This part of the brain assists in the process of accepting or rejecting an answer that comes to mind. The metadata on the modified trails B task is again very prominent in differentiating different cognitive groups. The longer average stroke length helps to distinguish those who have normal cognition (Task 1 and 2). The less straight the strokes were in the modified Trails B task and the longer time it took to complete the task, the more likely they had a dementia diagnosis (Task 3, 4, and 5). The length of time to draw a clock and the average clock draw stroke speed, the more likely they had a dementia diagnosis. Both the modified Trails B and the clock drawing tasks require executive and visuospatial skills and can be impaired by deficits in either or both domains. This may be why they appear in the top tier of behavioral features in differentiating between dementia and the other groups.

### Using Important Features Only

Table 7 compares the results when all the behavioral and scoring features (i.e., F-bs), and only the identified, top-5 important features from F-bs (in Table 4) are used. Among the 5 classification tasks, four tasks—Task 1 (CI vs. NC), Task 2 (MCI vs. NC), Task 4 (DM vs. MCI) and Task 5 (DM vs. nDM), had their AUC values even improved by using only the top-5 most important features instead of all F-bs features. In Task 1, the top-5 most important features included one behavioral feature and four scoring features (Table 4). The five features together improved the AUC to 0.9406 compared to 0.9288 from all the F-bs features. This may indicate that all the F-bs features together include redundant information or noises, but the top-5 most important features could synergistically carry the most useful information for the classification. Meanwhile, the rec. value dropped using the top-5 features only, with an increased prec., indicating that the model tended to be more conservative in predicting CI using the top-5 features only but can make its prediction more precisely. In Task 2, the top-5 most important features also included one behavioral feature (Table 5). With those five features, the performance for Task 2 was significantly improved on all the five metrics. Similarly, as in Task 1, this may be because that all the F-bs features together include redundancy or noises, but the top-5 most important features could capture the most informative signals to separate MCI and NC. For Task 4 and Task 5, their most important features were all scoring features (i.e., F-s), and in terms of AUC, F-s and F-bs did not show a (significant) difference (Table 3). However, when only the important scoring features were used, their AUC values were significantly improved. This may be due to the potential redundant information or noises in F-s features in detecting DM. For Task 3, using the most important features, where were all scoring features, did not help. This may indicate that for detecting DM from NC, only the top scoring features are not sufficient and other features would complement the needed information for the classification.

TABLE 7

| Task | Classes | Model | Features | acc | prec | rec | F1 | AUC |
|---|---|---|---|---|---|---|---|---|
| Task 1 | CI vs. NC | LR (l₁, l₂) | F-bs | 0.7869 | 0.8345 | 0.8911 | 0.8493 | 0.288 |
| | | LR (l₂) | top-5 | 0.7541 | 0.8524 | 0.7994 | 0.8147 | 0.9406 |
| Task 2 | MCI vs. NC | LR (l₁, l₂) | F-bs | 0.6606 | 0.7343 | 0.6700 | 0.6628 | 0.8870 |
| | | LR (l₂) | top-5 | 0.7522 | 0.8267 | 0.7100 | 0.7394 | 0.9175 |
| Task 3 | DM vs. NC | LR (l₂) | F-bs | 0.9600 | 1.0000 | 0.9200 | 0.9500 | 0.9920 |
| | | LR (l₂) | top-5 | 0.9300 | 0.9200 | 0.9600 | 0.9333 | 0.9720 |
| Task 4 | DM vs. MCI | LR (l₁, l₂) | F-bs | 0.7589 | 0.8033 | 0.7400 | 0.7100 | 0.9125 |
| | | LR (l₂) | top-5 | 0.7811 | 0.8433 | 0.7400 | 0.7259 | 0.9255 |
| Task 5 | DM vs. nDM | LR (l₁) | F-bs | 0.8672 | 0.9100 | 0.6900 | 0.7278 | 0.9426 |
| | | LR (l₂) | top-5 | 0.8344 | 0.8243 | 0.7300 | 0.7209 | 0.9586 |

### Using Modified Trails Average Stroke Length as a New Score

Since Modified Trails Average Stroke Length (in pixels) emerged among top most important features, its feasibility as an additional biomarker was further investigated. In evaluating the F1 values when only Modified Trails Average Stroke Length is used to classify CI vs. NC, if Modified Trails Average Stroke Length is shorter than a threshold, it is classified as CI. When using the Modified Trails Average Stroke Length between 900 and 1900 as the decision boundary, this feature alone is able to achieve an F1 value 0.8391 with a prec. 0.7333 and rec 0.9778. Compared with the performance for CI vs. NC classification in Table 3, the performance from this feature alone is close to the best performance (i.e., with F-bs, F1 0.8493, prec. 0.8345 and rec 0.8911). Particularly, the rec. value is even higher, indicating the effectiveness of this feature in identifying CI subjects.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, are hereby incorporated by reference in their entirety herein.
[1] Alzheimer's Association Report, 15(3), 321-387. doi:10.1016/j.jalz.2019.01.010
[2] Binaco, R., Calzaretto, N., Epifano, J., McGuire, S., Umer, M., Emrani, S., & Polikar, R. (2020). Machine Learning Analysis of Digital Clock Drawing Test Performance for Differential Classification of Mild Cognitive Impairment Subtypes Versus Alzheimer's Disease. Journal of the International Neuropsychological Society, 26(7), 690-700. doi:10.1017/S1355617720000144.
[3] Evans, D. A., Funkenstein, H. H., & Albert, M. S. (1989). Prevalence of Alzheimer's Disease in a Community Population of Older Persons: Higher Than Previously Reported. JAMA, 262(18), 2551-2556.
[4] Friedman, J. H. (1999). Greedy Function Approximation: A Gradient Boosting Machine. IMS 1999 Reitz Lecture.
[5] Grossberg, G. T. (2003). Diagnosis and Treatment of Alzheimer's Disease. J Clin Psychiatry, 64, 3-6.
[6] Lahmiri, S., & Shmuel, A. (2019). Performance of machine learning methods applied to structural MRI and ADAS cognitive scores in diagnosing Alzheimer's disease. Biomedical Signal Processing and Control, 52, 414-419. doi:10.1016/j.bspc.2018.08.009
[7] Pedregosa, F., Varoquaux, G., Gramfort, A., Michel, V., Thirion, B., Grisel, O., . . . Duchesnay, E. (2011). Scikit-learn: Machine Learning in Python. JMLR, 12, 2825-2830.
[8] Scharre, D. W., Chang, S. I., Murden, R. A., Lamb, J., Beversdorf, D. Q., Kataki, M., & Bornstein, R. A. (2010). Self-administered Gerocognitive Examination (SAGE): a brief cognitive assessment Instrument for mild cognitive impairment (MCI) and early dementia. Alzheimer Dis. Assoc. Disord., 24(1), 64-71.
[9] Scharre, D. W., Chang, S. I., Nagaraja, H. N., Vrettos, N. E., & Bornstein, R. A. (2017). Digitally translated Self-Administered Gerocognitive Examination (eSAGE): Relationship with its validated paper version, neuropsychological evaluations, and clinical assessments. Alzheimer's Research & Therapy, 9, 44.

What is claimed is:

1. A method to assess cognitive impairment or cognitive function, the method comprising:
obtaining, by one or more processors, a first data set comprising a set of question scores for a set of cognitive questions performed by a user;
obtaining, by the one or more processors, a second data set comprising at least one of a timing component log, a writing component log, and a drawing component log acquired during completion of the at least one of the set of cognitive questions by the user;
determining, by the one or more processors utilizing at least a portion of the first data set and second data set, one or more calculated values for at least one of a timing component feature, a writing component feature, and a drawing component feature for each of the at least one of the set of cognitive questions, wherein the drawing component feature includes at least one of a number of strokes, a total length of strokes, an average length of strokes per stroke, an average speed of strokes per stroke, an average straightness per stroke, a geometric area assessment of the strokes, or a geometric perimeter assessment of the strokes;
determining, by the one or more processors, based on the one or more calculated values for the at least one of the timing component feature, the writing component feature, and the drawing component feature, an estimated value for a presence of a cognitive disease or a score for cognitive level function; and
outputting, via a report and/or display, (i) the estimated value for the presence of the cognitive disease, condition, or an indicator of either or (ii) the score for cognitive level function, wherein the output is made available to a healthcare provider, a test evaluator, or a user to assist in a diagnosis of a cognitive disease or condition or a quantification of cognitive function.

2. The method of claim 1, wherein the estimated value for a presence of a cognitive condition or a score for the cognitive level function is determined using one or more trained ML models.

3. The method of claim 2, wherein the one or more trained ML models includes one or more logistic regression-associated models, one or more support vector machines, one or more neural networks, and/or one or more gradient boost-associated models.

4. The method of claim 1, wherein the estimated value for a presence of a cognitive condition or a score for the cognitive level function is determined using one or more trained AI models.

5. The method of claim 1, wherein the drawing component feature is determined from a time and position log of a user input to a pre-defined writing or drawing area during the completion of the at least one of the set of cognitive questions by the user.

6. The method of claim 5, wherein the drawing component feature is determined by a drawing component analysis module, the drawing component analysis module being configured by computer-readable instructions to:
i) identify, for each instance in the time and position log, an entry position and entry time for a given stroke and an exit position and an exit time for the given stroke and
ii) determine a measure from the entry position, entry time, exit position, and exit time for the given stroke.

7. The method of claim 6, wherein the measure includes at least one of:
i) determining the number of strokes;
ii) determining the total length of the strokes by (a) determining a length for each of the strokes and (b) summing the determined lengths;
iii) determine the average length of strokes per stroke by (a) determining a length for each stroke and (b) performing an average operation on the determined lengths;
iv) determining the average speed of strokes per stroke by (a) determining a velocity for each stroke using length and time measure for a given stroke and (b) performing an average operation on the determined lengths;
v) the average straightness per stroke by determining a ratio of a distance between each endpoint of the stroke to a corresponding length of the stroke; and
vi) determining a size of a response comprising the strokes.

8. The method of claim 7, wherein the measure of the average straightness per stroke is further determined by:
segmenting a single stroke of a geometric shape at corners of the geometric shape to generate individual strokes for each side of the geometric shape.

9. The method of claim 6, wherein the drawing component analysis module is configured to identify a number of extra strokes, wherein the extra strokes are not employed in the measure determination.

10. The method of claim 6, wherein the measure further includes handwriting analysis.

11. The method of claim 1, further comprising:
obtaining, by the one or more processors, a third data set comprising electronic health records of the user; and
determining, by the one or more processors utilizing a portion of the third data set, one or more calculated second values for a cognitive impairment feature,
wherein the one or more calculated second values for the cognitive impairment feature are used with the one or more calculated values for the drawing component feature to determine the estimated value for the presence of a cognitive disease or the score for cognitive level function.

12. The method of claim 11, wherein the first data and the second data are acquired through web services, and wherein the estimated value for the presence of the cognitive disease or cognitive level function are outputted through the web services to be displayed at a client device associated with the user.

13. The method of claim 12, wherein the output includes the estimated value for the presence or non-presence of the cognitive disease, condition, or an indicator of either, includes:
a measure for normal cognition, mild cognitive impairment (MCI), or dementia.

14. The method of claim 13, wherein the output includes the estimated value for the presence or non-presence of the cognitive disease, condition, or an indicator of either and is used by the healthcare provider to assist in the diagnosis of an early onset of Alzheimer's, dementia, memory loss, or cognitive impairment.

15. The method of claim 14, wherein the output includes the score for cognitive level function and is used by a test evaluator, in part, to evaluate the user in a job interview, a job-related training, or a job-related assessment.

16. A system comprising:
a processor; and
a memory having instructions stored thereon, wherein the instructions, when executed by a processor, cause the processor to
obtain a first data set comprising a set of question scores for a set of cognitive questions performed by a user;
obtain a second data set comprising at least one of a timing component log, a writing component log, and a drawing component log acquired during completion of the at least one of the set of cognitive questions by the user;
determine, utilizing at least a portion of the first data set and second data set, one or more calculated values for at least one of a timing component feature, a writing component feature, and a drawing component feature for each of the at least one of the set of cognitive questions, wherein the drawing component feature includes at least one of a number of strokes, a total length of strokes, an average length of strokes per stroke, an average speed of strokes per stroke, an average straightness per stroke, a geometric area assessment of the strokes, or a geometric perimeter assessment of the strokes;
determine, based on the one or more calculated values for the at least one of the timing component feature, the writing component feature, and the drawing component feature, an estimated value for a presence of a cognitive disease or a score for cognitive level function; and
output, via a report and/or display, (i) the estimated value for the presence of the cognitive disease, condition, or an indicator of either or (ii) the score for cognitive level function, wherein the output is made available to a healthcare provider, a test evaluator, or a user to assist in a diagnosis of a cognitive disease or condition or a quantification of cognitive function.

17. The system of claim 16 further comprising:
a cognitive test server configured to present, and obtain answers for, the set of cognitive questions to the user, wherein the cognitive test server is configured to generate a time and position log for one or more actions of the user when answering the set of cognitive questions.

18. A non-transitory computer-readable medium having instructions stored thereon, wherein the instructions, when executed by a processor, cause the processor to:
obtain a first data set comprising a set of question scores for a set of cognitive questions performed by a user;
obtain a second data set comprising at least one of a timing component log, a writing component log, and a drawing component log acquired during completion of the at least one of the set of cognitive questions by the user;
determine, utilizing at least a portion of the first data set and second data set, one or more calculated values for at least one of a timing component feature, a writing component feature, and a drawing component feature for each of the at least one of the set of cognitive questions, wherein the drawing component feature includes at least one of a number of strokes, a total length of strokes, an average length of strokes per stroke, an average speed of strokes per stroke, an average straightness per stroke, a geometric area assessment of the strokes, or a geometric perimeter assessment of the strokes;
determine, based on the one or more calculated values for the at least one of the timing component feature, the writing component feature, and the drawing component feature, an estimated value for a presence of a cognitive disease or a score for cognitive level function; and
output, via a report and/or display, (i) the estimated value for the presence of the cognitive disease, condition, or an indicator of either or (ii) the score for cognitive level function, wherein the output is made available to a healthcare provider, a test evaluator, or a user to assist in a diagnosis of a cognitive disease or condition or a quantification of cognitive function.

19. The system of claim 17, wherein the drawing component feature is determined by a drawing component analysis module, the drawing component analysis module being configured by computer-readable instructions to:
i) identify, for each instance in the time and position log, an entry position and entry time for a given stroke and an exit position and an exit time for the given stroke and
ii) determine a measure from the entry position, entry time, exit position, and exit time for the given stroke.

20. The system of claim 19, wherein the drawing component analysis module is configured to identify a number of extra strokes, wherein the extra strokes are not employed in the measure determination.
